(19) United States
(12) Patent Application Publication
(10) Pub. No.: US 2013/0096440 A1
(43) Pub. Date: Apr. 18, 2013

(54) PORTABLE FETAL EEG-RECORDING DEVICE AND METHOD OF USE

(71) Applicant: Marianna Kiraly, Daly City, CA (US)

(72) Inventor: Marianna Kiraly, Daly City, CA (US)

(21) Appl. No.: 13/396,233

(22) Filed: Feb. 14, 2012

Related U.S. Application Data
(60) Provisional application No. 61/627,626, filed on Oct. 14, 2011.

(51) Int. Cl. A61B 5/0205; A61B 8/02; A61B 5/0476

(52) U.S. Cl. 600/483; 600/544; 600/453

(57) ABSTRACT

A portable device utilizes an ultrasound probe, which is placed on the abdomen of a mother and allows ultrasound video and pictures, as well as Doppler heartbeat detection of the fetus. It is also capable of performing spontaneous brain activity recordings (referred here as fetal-EEG or fetal electroencephalography) and analyzing the fetal EEG measurements. The spontaneous electrical brain waves are detected by a sensor, amplified, digitized, and analyzed in one portable device, using analog and/or digital filters to improve the signal/noise ratio. The portable computer uses quantitative analysis software to compare the data from the fetus to normative data or to prior states of the fetus' own data. The EEG signals are recorded for an extended period of time and can be analyzed in real time or at a later time for rhythmicity patterns indicative of epilepsy or other developmental brain disorders.

### CLAIM FOR PRIORITY AND CROSS-REFERENCE TO RELATED APPLICATIONS

This application claims the priority of Provisional Patent Application Ser. No. 61/627,626, filed on Oct. 14, 2011 for Marianna Kiraly.

### FIELD OF THE INVENTION

The present invention relates to basic developmental neuroscience, portable medical obstetric procedures and devices, and more particularly to the non-invasive monitoring of the heart and brain physiology states of a developing human embryo inside the mother's womb.

### BACKGROUND OF THE INVENTION

Many of the developmental disorders of childhood—cerebral palsy, epilepsy, cognitive impairment from prematurity and autism—appear to result from an interaction of complex genetic traits and environmental factors. Likewise, adult psychiatric diseases may have their origins in impaired early, even fetal, development, as proposed for schizophrenia [1]. Despite major efforts, these prevalent, debilitating, life-long disorders remain biologically unexplained. Based on animal studies, the development of most types of epilepsy, cerebral palsy, autism and schizophrenia is suggested to link to neonatal seizures and various disturbances during embryogenesis. The intimate connection between mother, fetus and placenta, the vast array of neuroactive hormones expressed in the mother or in the placenta, or a variety of other environmental factors (injuries, drug treatments, immune responses, infections, hypoxic stress) make the targets when investigating fetal environmental disruptions that can affect the brain.

Perhaps one of the most important and challenging neuroscience research tasks would be to study the role of early brain activity in developmental plasticity and in the activity-dependent formation of neural circuits. It is evident that before birth, the immature brain expresses primitive, self-balancing rhythmic activity—with no complex excitatory and inhibitory synapses—in order to protect the developing brain from uncoordinated network activities or hyperexcitation. This early electrographic pattern was recorded in preterm human infants and newborn animals during sleep, immobility or feeding behavior. This primitive rhythm contains long silence periods, and is poor in information content, not necessarily associated with specific information—e.g. presence in the retina before eye opening—, but is indispensable to turn on the machine and ignite the network [2].

In previous human studies (for review see [3]) it was noted that the cortical EEG (electroencephalogram) recorded in neonates during the second half of gestation is organized in intermittent bursts that are separated by periods of virtually complete suppression of activity that could last for minutes. With maturation, suppression of activity between the bursts becomes less pronounced. At full-term, some discontinuity is still evident. At mid-gestation, the activity is dominated by delta waves of 0.3-2.0 Hz. By the seventh month of gestation, slow oscillations are intermixed with rapid rhythms. Each event of rapid activity consists of 8-25 Hz spindle-like, rhythmic activity superimposed on 0.3-1.5 Hz delta waves. These rhythms (referred as “delta brushes”) are predominantly expressed in central areas before 28 weeks, and are then recorded in central, temporal and occipital areas from 28 weeks to near term. Presence of delta brushes in EEG from preterm infants serves as a criterion of normal development, whereas their absence is indicative of brain pathology and poor prognosis. In addition to delta brushes, several other patterns have been described in premature neonates.

Even though these specific, immature rhythmic activity patterns could be perfect indicators to identify each stage of the healthy functional brain development, no existing device provides a sensitive enough detection and recording system able to conveniently perform its long-term time-dependent monitoring. In most cases, it is already too late after birth to permanently reverse the poor neurological outcomes, as they are being developed during embryogenesis due to microenvironmental alterations, which may change the process of neuronal migration and result in brain malformations or the establishment of incorrect or defective synaptic connections.

Recording of EEG signals is generally known in the medical arts. Use of ultrasound to display a fetus or to measure its Doppler cardiogram is also generally known in the medical arts. Further, recording of fetal brain wave signals is known in the prior art, for example in U.S. Pat. No. 6,556,861 to Prichep, and in U.S. Pat. No. 7,016,722 to Prichep. The entire disclosures of these two patents are expressly referred to and incorporated herein by reference thereto.

A flowchart of the device of U.S. Pat. No. 6,556,861 is shown herein as FIG. 1, which represents the prior art. The prior art steps shown in FIG. 1 are described in detail in that referenced patent specification, which as noted in the foregoing paragraph has been incorporated herein by reference.

It is a problem in the prior art to detect spontaneous brain activity in a developing fetus. As a consequence, it is also a problem in the prior art to detect signs of epilepsy or other brain injuries or disorders in a developing fetus, as in most cases these are not correlated with responses to auditory stimuli. There is accordingly a need in the prior art for a small, portable device (e.g. a smartphone-based instrument) that provides the convenience to a pregnant woman to perform such long-term measurements at home, anytime; and preferably after being trained in its use by a physician.

It is a further problem and need in the prior art to provide a portable fetal-EEG recording device that is extremely sensitive, detecting potentials of even below 1-2 microvolts, capable of detecting and recording signals over an extended period of time, and perform the steps of analyzing the recorded signals for signs of developmental brain disorders in the developing fetus.

It is also a problem in the prior art to provide visual control of the fetus, in order to determine the movement of the fetus between the time of application of the electrodes to the time of later measurements. This is intended to prevent occurrence of artifacts in the recordings. In accordance with the present invention, the brain waves of the fetus can not only be correlated to its position and activity, but also to its ECG (electrocardiogram) patterns or its Doppler-based heart rate, to better understand how its current brain activity changes during awake and sleep states.

### SUMMARY OF THE INVENTION

In the present invention, the spontaneous rhythmic brain activity of the fetus is non-invasively detected and analyzed in a portable fetal-EEG recording device. This is a procedure performed for an extended period of time using sensitive but comfortable, lightweight equipment (preferably a hand-held device). Recording such fetal-EEG signals is of great importance, as these can serve as indicators for certain unhealthy conditions or environmental factors (e.g. altered maternal hormone levels, stress, drug treatment, etc.) that may risk the normal brain development of the fetus. The identification and exclusion of such factors and conditions during embryogenesis may help to avoid the development and progression of several neural disorders that are already untreatable after birth.

One or a grid of detecting sensor electrodes is removably attached to the abdominal skin of the pregnant woman, in close proximity to the head and/or heart of the fetus. The electrical connectivity between the sensor and the abdominal skin can be improved by using an adhesive gel enriched with electrolytes.

The sensor electrode connected to the fetal-EEG recording device is capable of detecting microvolt level fetal brain activity patterns, which can be recorded using similar low-noise (<1 microvolt) amplification (preferred gain≧200000) and optional bandpass filtering methods as known to be used for neurophysiology research purposes.

In order to better understand the measured data, an ultrasound probe (operated at 3.5-5 MHz frequency) can be connected to the “portable fetal-EEG recording device”, and the position of the fetus can be real-time monitored on the display of the device, in order to avoid the misinterpretation of data caused by movement of the fetus subsequent to the application of the electrodes resulting in incorrect readings, and which could therefore cause certain movement artifacts. The same or another ultrasound probe (operated at 2-3 MHz) connected to the same device may serve as a Doppler heart monitor for the fetus. When placing one of the sensor electrodes in close proximity to the heart of the fetus, it may serve as an ECG electrode. The simultaneous use of the Doppler ultrasound probe and ECG electrode may help the user make sure that the operation mode of the device is correct and both of the methods work properly. Monitoring the fetal heart frequency may provide additional information about the current activity of the fetus (e.g. allow the determination of its awake and sleep states).

Further computational (software) filtration and analysis of all electrical recordings can be performed in accordance with those known from conventional routine clinical EEG-recording methods. This may help in identifying electrical artifacts, as well as noise derived from the heart or muscles of the fetus or mother (e.g. fetal eye-movements). The portable fetal-EEG recording device provides an output for an Internet connection, and therefore allows all of the recorded ultrasound images and videos, raw and analyzed EEG recordings to be broadcasted in real-time, or later shared with the obstetrician/gynecologist, pediatric neurologist or any friends or family members of the user.

### BRIEF DESCRIPTION OF THE DRAWINGS

FIG. 1 shows a flowchart for a prior art device according to U.S. Pat. No. 6,556,861, and is described therein.

FIG. 2 is a simple schematic demonstration of the noninvasive fetal-EEG device, ultrasound module, and electrodes according to the present invention.

FIG. 3 is a flowchart depicting steps in the use of device and method according to the present invention.

FIG. 4 is a schematic view of a portable device according to the present invention, showing structural features thereof and connections with an ultrasound device and a fetal-EEG recording device.

FIG. 5 is a schematic view of a portable device according to the present invention, showing functional connections and output features thereof, as well as connections with an ultrasound device, a fetal Doppler signal detecting device, and a fetal-EEG recording device.

FIG. 6 is a schematic flowchart of steps showing use of the invention of FIGS. 2-5.

### DETAILED DESCRIPTION OF THE INVENTION AND METHOD

The present invention, discussed in detail hereunder, relates to a portable device and a method for using the portable device to detect fetal heart rate and EEG signals, and to detect signs of normal and abnormal embryonic development. The device of the present invention provides an Internet connection, and it serves as an apparatus for performing and analyzing fetal-EEG and ECG recordings, ultrasound imaging and Doppler heartbeat detection.

Technologies which can be used in the present invention, and which are commercially known and available for use, are known in the art and samples of these are as follows. The type of electrodes and method of use feasible for the present invention are known, for example in U.S. Pat. No. 6,162,101 issued on Sep. 3, 1998 to Fisher and Iversen; U.S. Pat. No. 6,024,702 issued on Feb. 3, 1997 to Iversen; U.S. Pat. No. 5,961,909 issued on Sep. 3, 1997 to Iverson; U.S. Pat. No. 5,902,236 issued on Sep. 3, 1997 to Iversen; as well as in other patent documents. The possibility of recording spontaneous electrical brain and heart activity of a fetus in utero has also been published [4, 5].

A portable abdominal ultrasound unit capable of performing advanced ultrasound measurements for obstetrical use is known and commercially available. Portable scalp-EEG recording instruments and portable Doppler devices capable of determining fetal heart rate have been commonly used and commercially available for a long time.

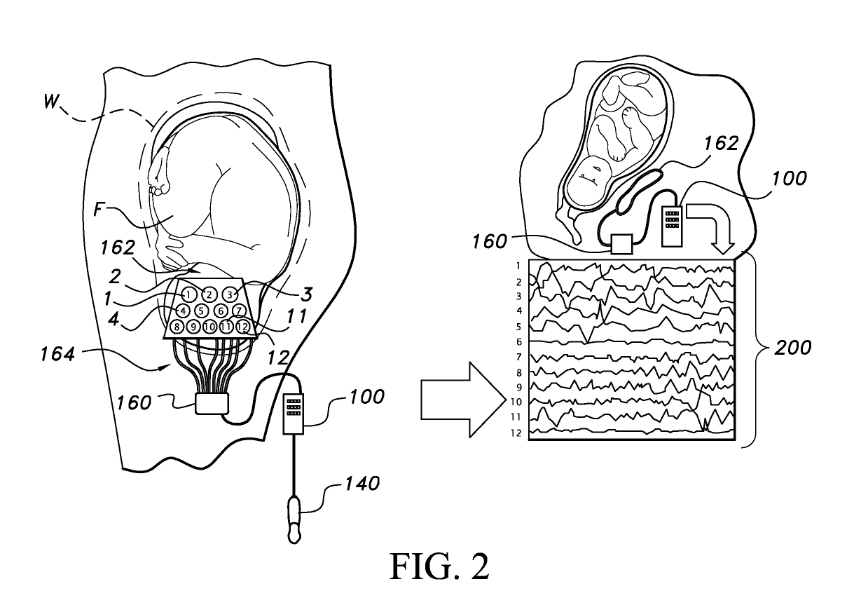

FIG. 2 is a simple schematic demonstration of a portable device 100, which is shown in FIG. 2 as being connected by any communication means including Bluetooth, Wireless Internet or wires to a fetal EEG detecting device 160 and to an ultrasound module 140. The fetal-EEG detecting device 160 is connected to an electrode or electrode sheet 162 by a plurality of connecting wires 164. The biosensor electrode or electrode sheet 162 has a plurality of sensitive electrodes thereon, of the type mentioned hereinabove, having a reference and multiple detector electrodes 1, 2, 3, 4, . . . , 11, and 12. The electrodes 1, 2, 3, 4, . . . , 11, and 12 can be numbered differently, and can be arranged in other types of geometrical locations, without departing from the scope of the present invention. The biosensor electrode or electrode sheet is removably attached to the patient's skin; it may also use conductive gel, providing rapid attachment and acceptably low noise.

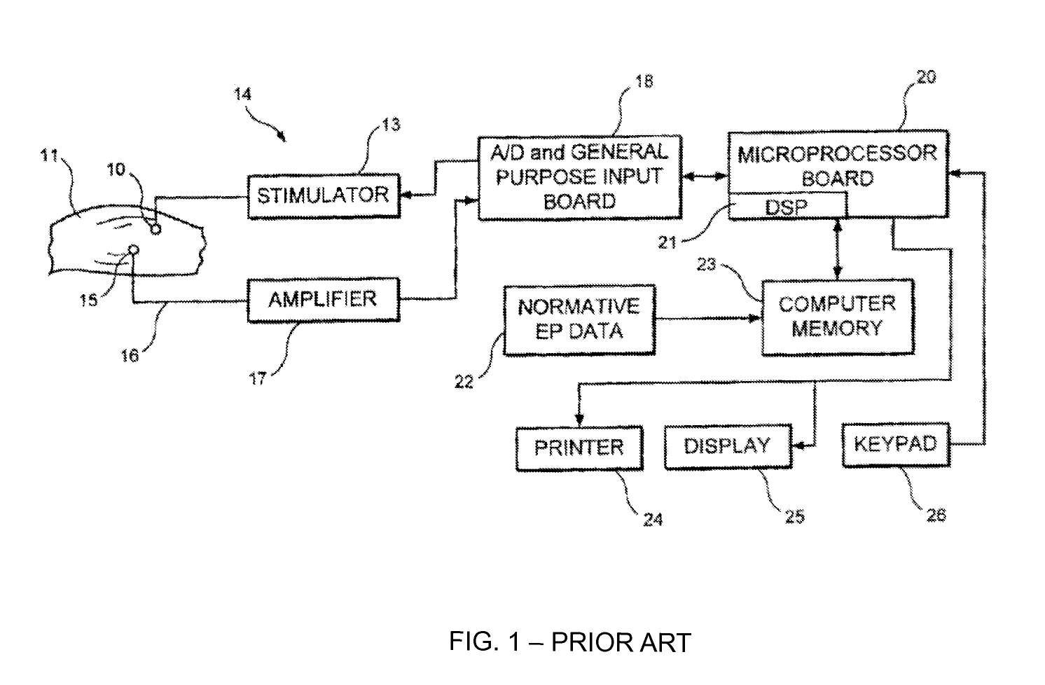

The ultrasound module 140 is an abdominal probe operated at 3.5-5 MHz in order to determine the position of the fetus, is operated by a special imaging software capable of recording high-resolution videos and images, and can be any commercially available ultrasound device compatible with the present invention. Optionally, it may be capable of Doppler heartbeat detection (operated at a range of 2-3 MHz). The fetal EEG detecting device 160 can be that shown in the above-mentioned prior art excluding the stimulator unit (FIG. 1), or can be a commercially available or custom developed device. These and other variations are all contemplated as being within the scope of the present invention.

In FIG. 2, the non-invasive fetal-EEG device is shown overlying a head and/or heart of a fetus F within a uterus W shown on the left hand side of FIG. 2. The right hand side of FIG. 2 depicts a schematic side view of the mother's uterus and the fetus, showing the electrode/electrode sheet 162 disposed on the mother's abdominal region directly over the head and/or heart of the fetus. Illustrated output signals from the detector electrodes 1, 2, 3, 4, . . . , 11, and 12 are shown at the output graph 200. These brain waves and/or ECG signals are recorded at a preferred sampling frequency of minimum 4 kHz, digitized, amplified using high input impedance of at least 1 MegaOhm, low-noise (<1 microvolt) amplification (preferred gain≧200000), and may preferably be filtered at a bandpass frequency of 0.5 to 40 Hz. The range of 0.5 to 40 Hz is typical, but not limiting; and a range up to 70 Hz is contemplated as being useable in the present invention. In addition, the recorded traces may also be integrated, in order to make the mathematical (statistical) analysis and peak-detection easier, and to provide a simple way for measuring wave amplitude, duration, integrated brain wave area, burst frequency or other quantitative parameters. The output graph 200 is by way of illustration only; in actual use a graph is not displayed but instead the signals are monitored and recorded continuously and over a relatively long period of time, e.g. hours or days. The signals can be processed either in real time or at a later time by specific recording and analysis software, and can be transmitted by the portable device 100 using the internet or using cell phone transmission, etc., to a computer for analyzing the signals, or to an obstetrician or other professional. Further software analysis and corrections provide additional noise reduction, and may help eliminate movement artifacts (i.e., where movement of the fetus occurs after placement of the electrodes resulting in incorrect output readings) or other non-specific signals (e.g. muscle activity, eye movement, mother's heart signals, etc.).

The portable device 100 combined with the fetal EEG detecting device 160 and the ultrasound module 140, constitutes a small, compact and portable EEG monitoring system, which can make it possible for physicians to follow the maturation of fetal brain activity in a real-time manner during high-risk pregnancies, maternal infections, hypoxia, stress, or other conditions. Qualitative and quantitative data evaluation methods described in the prior art and studies [6, 7] can be applied to determine the functional developmental status of the fetus. The raw and analyzed spontaneous fetal EEG data can be compared to reference spontaneous fetal EEG data from a control group to determine one of an abnormality and normality of the brain activity and heart rate of the fetus being monitored. However, in the lack of a proper instrument capable of detecting human fetal brain waves in utero, yet little is known about the brain activity of unborn human fetuses. Therefore this present invention will be a useful tool for scientific research purposes, in order to better address and understand the functional brain development process of human embryos.

The small, portable EEG-device 100 of the present invention is capable of recording data all day long, causing no inconvenience in continuing the usual activities of the user's everyday life. The registered waves can be analyzed either real-time, or later in the office of a gynecologist or pediatric neurologist. This technology can be applied in construction of the portable device 100 of the present invention, which is thereby made as a small, user-friendly and affordable fetal-EEG device specifically designed for clinical purposes, which will be ideal for everyday usage and reliable diagnostics.

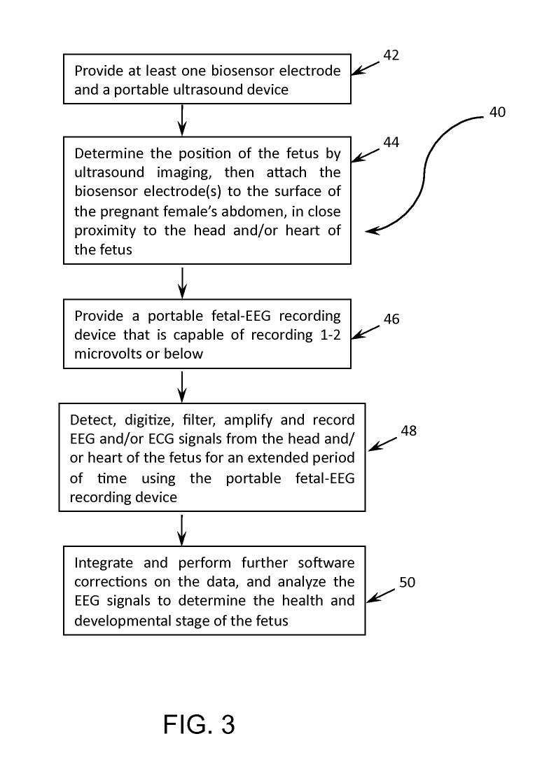

FIG. 3 is a flowchart 40 depicting steps 42, 44, 46, 48, and 50 in the use of device and method according to the present invention.

The steps include (step 42) providing a biosensor electrode, or an electrode grid or sheet and a portable ultrasound device, then determining the position of the fetus (step 44) and attaching the sensor or electrode sheet having the EEG electrodes to the surface of the abdomen right above the head and/or heart of the fetus.

Following the above steps 42 and 44, further providing (step 46) a portable fetal-EEG recording device (such as portable device 100 described hereinabove with reference to FIG. 2) that is extremely sensitive, detecting potentials of 1-2 microvolts or below, and then (step 48) recording the EEG and/or ECG signals from the head and/or heart of the fetus for an extended period of time using the portable fetal-EEG recording device. It is recommended to repeatedly determine the position of the fetus by ultrasound imaging, in order to determine the movement of the fetus between the time of application of the electrodes to the time of later measurements. This is intended to prevent occurrence of artifacts in the recordings.

Following the above steps 42, 44, 46, and 48, further analyzing (step 50) the recorded fetal-EEG signals for signs of neural network activity patterns indicative of brain disorders, including the steps of digitizing the signals, filtering the signals from all non-specific noise, amplifying the signals, integrating the signals, and storing the signals in a relatively small portable storage medium.

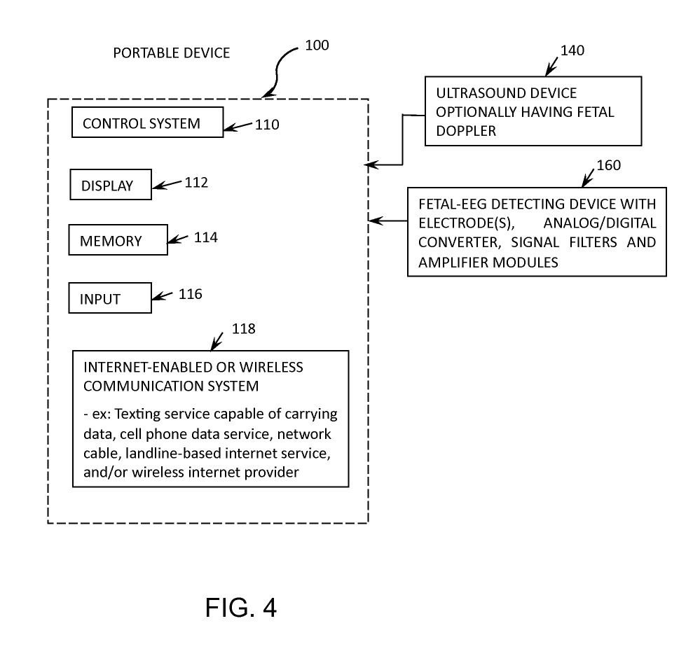

FIG. 4 is a schematic view of the portable device 100 according to the present invention, showing structural features thereof and connections with an ultrasound device 140 and a fetal-EEG recording device 160.

As seen in FIG. 4, the portable device 100 is shown in dashed outline, and preferably includes a control system 110, a display 112, a memory 114, an input means 116 (such as a touch pad, a keyboard, a mouse, or other input devices), and an internet-enabled or wireless communication system 118. The internet-enabled or wireless communication system 118 can be of a type already known in smartphone technologies, or it can be a custom-built portable device within the ambit of skill of any one having skill in the smartphone arts. The elements 110, 112, 114, and 116 can all be types which are present in existing smartphone technologies, or can be custom made within the ambit of skill of any one having skill in the smartphone arts and/or the smartphone application programming arts.

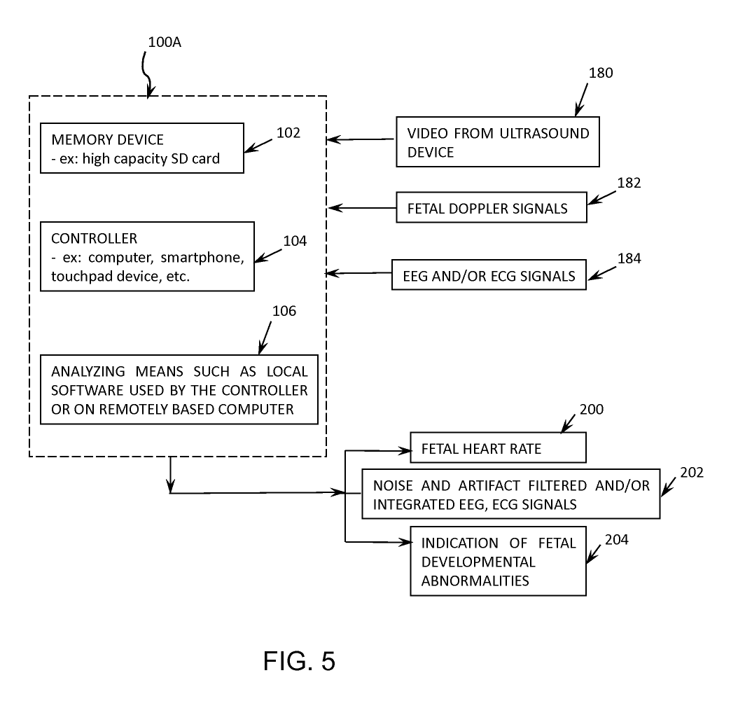

FIG. 5 is a schematic view of a portable device 100A according to the present invention, showing functional connections and output features thereof, as well as connections with an ultrasound device 180, a fetal Doppler signal detecting device 182, and a fetal-EEG recording device 184 which records EEG and/or ECG signals from electrodes. The portable device 100A can be similar or identical to the portable device 100 shown and discussed hereinabove, or it can be a variation of that device.

The portable device 100A includes a memory device 102 which can, for example, be a high capacity SD card or other type of memory device. The portable device 100A also includes a controller 104 which can, for example, be a computer or computer chip, a smartphone, smart touchpad device having computer technology, etc.

The portable device 100A also includes an analyzing function means 106 such as local software used by the controller 104, or else supplies data to a remotely based computer for software analysis using the internet or cell phone technology.

The portable device 100A provides outputs, which can include fetal heart rate 200, noise and artifact filtered EEG, ECG and/or integrated EEG signals 202, and an indication of fetal developmental abnormalities such as intrauterine seizures or other abnormal brain activity 204. These signals can be obtained using the software, and the detection and determination of normal and abnormal human fetal brain activity is an evolving field. It is anticipated that future discoveries may be made in this evolving field, and it is contemplated that the results of such discoveries can be used in the indication of abnormal fetal development 204.

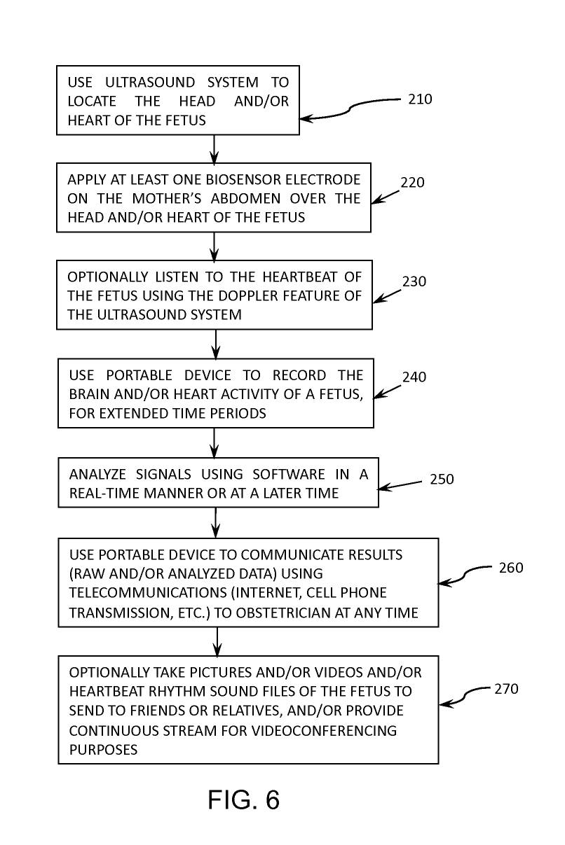

FIG. 6 is a schematic flowchart of steps showing use of the invention of FIGS. 2-5. Here, step 210 is use of the ultrasound system 180 to locate the head and/or heart of the fetus. Then at step 220, the electrode sheet 162 is applied to the mother's abdominal region over the head and/or heart of the embryo. Step 230 is optional, listening to the heartbeat of the fetus using the Doppler feature of the fetal Doppler signals from the ultrasound device 182. Step 240 is using the portable device 100 or 100A to record the brain and/or heart activity of the fetus (using the signals received from the electrode or electrode sheet 162) for extended time periods.

Also in FIG. 6, the step 250 is analyzing the above-mentioned detected signals using software in a real time manner or at a later time. Step 260 is using the portable device 100 or 100A to communicate results (raw and/or analyzed data) using telecommunication means as discussed hereinabove (e.g. internet, cell phone transmissions, etc.) to an obstetrician or other professionals at any time. The Step 260 also is contemplated to include transmitting stored data saved over a relatively long period of time, and having that data analyzed by remote software, by an obstetrician, or by other professionals at any time.

Lastly, the optional step 270 is using the ultrasound device 180 to take pictures and/or videos and/or sound files of the baby to send to relatives, friends, and/or medical professionals, and/or to provide a continuous stream of video for webcam or videoconferencing purposes.

The invention being thus described, it will be evident that the same may be varied in many ways. Such variations are not to be regarded as a departure from the spirit and scope of the invention and all such modifications are intended to be included within the scope of the claims.

### REFERENCES

1. Meyer U, Yee B K, Feldon J.: The neurodevelopmental impact of prenatal infections at different times of pregnancy: the earlier the worse? Neuroscientist. 2007 June; 13 (3):241-56.

2. Ben-Ari Y, Gaiarsa J L, Tyzio R, Khazipov R: GABA: a pioneer transmitter that excites immature neurons and generates primitive oscillations. Physiol Rev. 2007 October; 87 (4):1215-84.

3. Khazipov R, Luhmann H J. Early patterns of electrical activity in the developing cerebral cortex of humans and rodents. Trends Neurosci. 2006 July; 29 (7):414-8. Epub 2006 May 19.

4. Khandoker, A. H.; Kimura, Y.; Palaniswami, M.; Marusic, S.: Identifying fetal heart anomalies using fetal ECG and Doppler cardiogram signals. Computing in Cardiology, 2010 September; 891-4.

5. Lindsley D B.: Heart and brain potentials of human fetuses in utero. Am J Psychol. 1987 Fall-Winter; 100 (3-4):641-6.

6. Scher M S, Turnbull J, Loparo K, Johnson M W.: Automated state analyses: proposed applications to neonatal neurointensive care. J Clin Neurophysiol. 2005 August; 22 (4):256-70.

7. Vanhatalo S, Kaila K.: Development of neonatal EEG activity: from phenomenology to physiology. Semin Fetal Neonatal Med. 2006 December; 11 (6):471-8. Epub 2006 Oct. 2.

What is claimed is:

1. A method of recording spontaneous electrical brain activity of a fetus in utero, comprising the steps of:
(a) identifying the head and/or heart of the fetus using an abdominal ultrasound probe connected to the portable fetal-EEG recording device, by real-time watching the ultrasound images and videos displayed by the device;
(b) removably connecting at least one sensor electrode to the mother's abdomen above the head and/or heart of the fetus to detect spontaneous brain activity in the fetus;
(c) providing a portable fetal-EEG recording device that is extremely sensitive and capable of detecting potentials of 1-2 microvolts or below;
(d) detecting the EEG signals from the head and/or heart of the fetus for an extended period of time using the portable fetal-EEG recording device, including the steps of digitizing the signals, optionally filtering the signals from non-specific noise, amplifying the signals, and storing the signals in a relatively small portable storage medium capable of connecting to the internet;
(e) analyzing the recorded fetal-EEG signals for network activity patterns indicative of epilepsy or other developmental brain disorders; and
(f) displaying the raw signals and the results of the analysis.

2. The method of recording as claimed in claim 1, further comprising the step of integrating the signal using a portable computer-based software or an integrator module.

3. The method of recording as claimed in claim 1, further comprising the step of improving a signal to noise ratio of the recorded brain waves using portable computer-based analysis software.

4. The method of recording as claimed in claim 1, further comprising the step of displaying results of the analysis as an indication of the status of brain function of the fetus.

5. The method of recording as claimed in claim 1, wherein, in step (a), the ultrasound probe is also capable of generating Doppler signals, which is used to produce an output representing fetal heart rate.

6. The method of recording as claimed in claim 1, wherein, in step (b), the at least one sensor electrode comprises an array of sensor electrodes attached to the mother's abdomen, and one electrode in the array is used as a reference electrode.

7. The method of recording as claimed in claim 1, wherein in step (b), the at least one sensor electrode is used produce an output representing a fetal electrocardiogram (ECG).

8. The method of recording as claimed in claim 1, further comprising the step of passing the recorded analog data through a band pass filter having a plurality of band pass frequency ranges within an overall frequency range of 0.5 to 40 Hz.

9. The method of recording as claimed in claim 1, further comprising the step of comparing the spontaneous fetal EEG data to reference spontaneous fetal EEG data from a control group to determine one of an abnormality and normality of the brain activity and heart rate of the fetus being monitored.

10. The method of recording as claimed in claim 1, further comprising the step of transmitting any of the images, videos or recordings via the internet.

11. A portable fetal-EEG recording device for recording EEG signals from a fetus in utero, comprising:
(a) an ultrasound probe connected to the portable fetal-EEG device, adapted to be placed on an abdomen of a mother of the fetus, to identify the position of the fetus and to localize its head and/or heart; and said ultrasound probe including computing means running an imaging software to real-time visualize the ultrasound images of the fetus;
(b) at least one sensor electrode adapted to be placed on the mother's abdomen for detecting spontaneous electrical activity of the brain of a fetus;
(c) an amplifier-filter module connected to the at least one sensor electrode to amplify the spontaneous brain activity of the fetus detected by the at least one biosensor electrode;
(d) an analog/digital converter converting the analog data to digital data;
(e) a portable computer-based quantitative analysis software improving a signal to noise ratio of the digitized spontaneous brain activity data and analyzing the data;
(f) a display to real-time demonstrate the raw data and the results of the analysis as an indication of a status of the fetus; and
(g) portable computer-based memory to store the data, and being capable of outputting said data for transmission to an external device or network.

12. The portable fetal-EEG recording device as claimed in claim 11, wherein the at least one sensor electrode includes an electrode grid, and one electrode in the grid is used as a reference electrode.

13. The portable fetal-EEG recording device as claimed in claim 11, wherein the computer system includes a filter having a plurality of band pass frequency ranges in an overall frequency range of 0.5 to 40 Hz.

14. The portable fetal-EEG recording device as in claimed in claim 11, wherein the amplifier-filter module (c) includes an integrator module.

15. The portable fetal-EEG recording device as claimed in claim 11, further comprising: an arrangement comparing the digitized spontaneous fetal brain activity data to comparative digitized spontaneous fetal brain activity data from a normal group of fetuses or the fetus' previously recorded own data to determine one of an abnormality and normality of the EEG/ECG signals.

16. A portable fetal-EEG recording device as claimed in claim 11, further comprising an input representing fetal Doppler signals, and wherein said control system uses the fetal Doppler signals to produce an output representing fetal heart rate.

17. A portable fetal-EEG recording device as claimed in claim 12, further comprising software used by said control system for analyzing the recorded EEG and ECG signals and producing an output representing the states of the fetus, namely sleep, awake or other states, and indicate any of fetal seizures and other abnormal brain activities.

18. A portable fetal-EEG recording device as claimed in claim 11, wherein the control system is extremely sensitive and capable of detecting potentials of 1-2 microvolts or below, at a preferred sampling frequency of approximately a minimum of 4 kHz.

19. The method of recording as claimed in claim 1, further comprising the step of transferring the images, videos, raw and analyzed data files via the Internet.
